(19) United States
(12) Patent Application Publication
(10) Pub. No.: US 2005/0053619 A1
(43) Pub. Date: Mar. 10, 2005

(54) Composition and method for providing temporary relief of sinus pain and nasal congestion

(76) Inventors: Samuel Berkowitz, Teaneck, NJ (US); Chaim Lieberman, Brooklyn, NY (US)

Correspondence Address: Dr. O. M. (Sam) Zaghmout;Bio Intellectual Property Services LLC, 8509 Kemon CT, Lorton, VA, 22079, US

(21) Appl. No.: 10/934,699

(22) Filed: Sep. 7, 2004

Related U.S. Application Data
(60) Provisional application No. 60/500,354, filed on Sep. 5, 2003.

(51) Int. Cl. A61K 35/78

(52) U.S. Cl. 424/195.18; 424/742; 424/747; 424/769; 424/770

(57) ABSTRACT

A composition for providing temporary relief of sinus pain and nasal congestion uses natural products. The composition is preferably administered nasally.

### PRIORITY

This application claims the benefit of Patent provisional application Ser. No.: 60/500,354, filed Sep. 5, 2003 by the present inventors.

### BACKGROUND OF THE INVENTION

1. Field of Invention

This invention relates to methods and compositions for reducing and alleviating respiratory problems. Particularly, this invention relates to methods and compositions for providing a temporary relief of sinus pain and nasal congestion. More particularly, this invention relates to the composition comprising an extract, which provides a temporary relief of sinus pain and nasal congestion. The composition of this invention may comprise a pharmaceutically acceptable carrier suitable for the chosen method and form of administration. In these embodiments, the composition may comprise a suitable carrier, and other ingredients such as flavors, stabilizers, lubricants, and additional natural or artificial sweeteners.

2. General Background

In the United States, it is estimated that a large percent of the population is afflicted with sinus pain and nasal congestion problems of sufficient severity to disturb the regular life of the person. Whatever the cause, the sinus pain and nasal congestion related problems produced are highly undesirable.

In the past, therapy for eliminating or reducing sinus pain and nasal congestion has included the administration of decongestants and antihistamines, which generally have been ineffective. Such medications have exhibited, at best, limited success for reducing drainage due to colds, allergies and the like.

The costs of treating sinus pain and nasal congestion with over-the-counter medications in the United States is estimated at an annual cost of over 1.5 billion dollars. The direct costs of treatment in outpatient clinics is estimated at almost four billion dollars, Indirect costs, based on the amount of loss in wages because of restricted activity are substantially higher. Accordingly, it is desirable to provide a simple, easy to use, relatively inexpensive, and effective procedure for substantially reducing or eliminating sinus pain and nasal congestion.

Accordingly, it is desirable to provide a simple, easy to use, relatively inexpensive, and effective composition and procedure for substantially reducing or eliminating sinus pain and nasal congestion.

### SUMMARY OF THE INVENTION

It is an object of this invention to provide a composition for reducing sinus pain and nasal congestion. It is an additional object of this invention to provide an improved natural remedy for reducing sinus pain and nasal congestion.

It is another object of this invention to provide an improved composition utilizing plant products for reducing sinus pain and nasal congestion.

In accordance with a preferred embodiment of this invention, a decongestant composition, which includes a set of active components, comprising Natrum muriaticum, Natrum muriaticum, Camphora, Mentholum and Eucalyptol. In a more preferred embodiment of this invention, the total active components is about 28% by weight or volume of said decongestant preparation. In a more preferred embodiment of this invention, the active components are about:
17.23% Natrum muriaticum
3.78% Camphora
3.78% Mentholum
3.78% Eucalyptol
   by weight or volume of said decongestant preparation.
   In a more preferred embodiment of this invention, the decongestant composition may contain one or more, additional, components. In a more preferred embodiment of this invention, the decongestant composition contains as additional components at least one member selected from the group consisting of Natural Sweet Birch Oil, Benzoin Resin, Thymol, Natural Pine Oil, Cinnamon Oil, Spearmint Oil, Sodium Bicarbonate, and Water. In a more preferred embodiment of this invention, the components are present in an amount of about:
   0.002212% Natural Sweet Birch Oil
   0.001106% Benzoin Resin
   0.001841% Thymol
   0.0005687% Natural Pine Oil
   0.0003782% Cinnamon Oil
   0.0032999% Spearmint Oil
   0.001116% Sodium Bicarbonate
   71.41% Water
      by weight or volume of said decongestant preparation

In accordance with a preferred embodiment of this invention, a method of treating the occurrence of sinus pain and/or nasal congestion, comprising the step of:

administering to the subject a pharmaceutically effective amount of a composition comprising:

Natrum muriaticum
Camphora,
Mentholum
Eucalyptol
Natural Sweet Birch Oil
Benzoin Resin
Thymol
Natural Pine Oil
Cinnamon Oil
Spearmint Oil
Sodium Bicarbonate
Water
   wherein the subject is a mammal, for example, wherein the subject is a human.

In accordance with a preferred embodiment of this invention, a method of inhibiting the occurrence of sinus pain and/or nasal congestion, comprising the step of: administering to the subject a pharmaceutically effective amount of a composition comprising:
Natrum muriaticum
Camphora,
Mentholum
Eucalyptol
Natural Sweet Birch Oil
Benzoin Resin
Thymol
Natural Pine Oil
Cinnamon Oil
Spearmint Oil
Sodium Bicarbonate
Water
   wherein subject is a mammal, wherein the subject is a human.

In accordance with a preferred embodiment of this invention, a method is disclosed for reducing or alleviating respiratory problems comprising the step of: administering to the subject a pharmaceutically effective amount of a composition comprising:
Natrum muriaticum
Camphora,
Mentholum
Eucalyptol
Natural Sweet Birch Oil
Benzoin Resin
Thymol
Natural Pine Oil
Cinnamon Oil
Spearmint Oil
Sodium Bicarbonate
Water
   wherein subject is a mammal, for example, wherein the subject is a human, wherein the respiratory problem is a sinus pain or a nasal congestion.

In accordance with a preferred embodiment of this invention, a composition for reducing sinus pain and nasal congestion and alleviating respiratory problems is comprised of compounds of Sinusol that are listed in Table (1).

The composition of this invention is preferably administered nasally. The composition may be administered, but not limited to, as extract, as liquid extract in liquid, as capsules or as tablets, with doses administered according to the weight of the user.

### DETAILED DESCRIPTION

It is an object of this invention to provide a composition for reducing sinus pain and nasal congestion. It is an additional object of this invention to provide an improved natural remedy for reducing sinus pain and nasal congestion.

It is another object of this invention to provide an improved composition utilizing plant products for reducing sinus pain and nasal congestion.

In accordance with a preferred embodiment of this invention, a decongestant composition comprising a set of active components comprising Natrum muriaticum, Natrum muriaticum, Camphora, Mentholum and Eucalyptol. In a more preferred embodiment of this invention, the total active components is about 28% by weight or volume of said decongestant preparation. In a more preferred embodiment of this invention, the active components are about:
17.23% Natrum muriaticum
3.78% Camphora
3.78% Mentholum
3.78% Eucalyptol
   by weight or volume of said decongestant preparation.

In a more preferred embodiment of this invention, the decongestant composition contains as additional one or more components. In a more preferred embodiment of this invention, the decongestant composition contains as additional components at least one member selected from the group consisting of Natural Sweet Birch Oil, Benzoin Resin, Thymol, Natural Pine Oil, Cinnamon Oil, Spearmint Oil, Sodium Bicarbonate, and Water. In a more preferred embodiment of this invention, the components are about:
0.002212% Natural Sweet Birch Oil
0.001106% Benzoin Resin
0.001841% Thymol
0.0005687% Natural Pine Oil
0.0003782% Cinnamon Oil
0.0032999% Spearmint Oil
0.001116% Sodium Bicarbonate
71.41% Water
   by weight or volume of said decongestant preparation.

In accordance with a preferred embodiment of this invention, a method of treating the occurrence of sinus pain and/or nasal congestion, comprising the step of:

administering to the subject a pharmaceutically effective amount of a composition comprising:

Natrum muriaticum
Camphora,
Mentholum
Eucalyptol
Natural Sweet Birch Oil
Benzoin Resin
Thymol
Natural Pine Oil
Cinnamon Oil
Spearmint Oil
Sodium Bicarbonate
Water
   wherein subject is a mammal, for example, wherein the subject is human.

In accordance with a preferred embodiment of this invention, a method of inhibiting the occurrence of sinus pain and/or nasal congestion, comprising the step of:

administering to the subject a pharmaceutically effective amount of a composition comprising:

Natrum muriaticum
Camphora,
Mentholum
Eucalyptol
Natural Sweet Birch Oil
Benzoin Resin
Thymol
Natural Pine Oil
Cinnamon Oil
Spearmint Oil
Sodium Bicarbonate
Water
   wherein subject is a mammal, for example, wherein the subject is a human.

In accordance with a preferred embodiment of this invention, a method is disclosed which is useful for reducing or alleviating respiratory problems comprising the step of:

administering to the subject a pharmaceutically effective amount of a composition comprising:

Natrum muriaticum
Camphora,
Mentholum
Eucalyptol
Natural Sweet Birch Oil
Benzoin Resin
Thymol
Natural Pine Oil
Cinnamon Oil
Spearmint Oil
Sodium Bicarbonate
Water
   wherein the subject is a mammal, for example, wherein the subject is a human, and wherein the respiratory problem is a sinus pain or a nasal congestion.

In accordance with a preferred embodiment of this invention, a composition for reducing sinus pain and nasal congestion and alleviating respiratory problems is comprised of compounds of Sinusol that are listed in Table (1).

In accordance with a preferred embodiment of this invention, a composition for reducing sinus pain and nasal congestion comprising plant products and extracts was discovered (Table 1). The invention is not limited to various ranges of formulation as listed in Table (1). The products are preferably combined and are placed in a liquid base for nasal administration or administration in a spray or mist form.

TABLE 1

| ORIGINAL | | | POTENTIAL | ml or g |
|---|---|---|---|---|
| | | | Actives | |
| Sodium | 0.001723 | g | Natrum muriaticum | 17.23 |
| Camphor | 0.003782 | g | Camphora 3X | 3.78 |
| Menthol | 0.003782 | g | Mentholum 3X | 3.78 |
| Eucalyptol | 0.003782 | g | Eucalyptol 3X | 3.78 |
| | | | Total Actives | 28.58 |
| | | | Inactives | |
| Natural Sweet | 0.002212 | g | Natural Sweet Birch | 0.002212 |
| Birch | | | Oil | |
| Benzoin Resin | 0.001106 | g | Benzoin Resin | 0.001106 |
| Thymol | 0.001841 | g | Thymol | 0.001841 |
| Natural Pine Oil | 0.0005687 | | Natural Pine Oil | 0.0005687 |
| Natural | 0.0003782 | | Cinnamon Oil | 0.0003782 |
| Spearmint Oil | 0.0032999 | | Spearmint Oil | 0.0032999 |
| Alcohol | 3.75 | g | Ethanol | 0 |
| Sodium | 0.001116 | g | Sodium | 0.001116 |
| Bicarbonate | | | Bicarbonate | |
| Balance Water | 96.23 | g | Water | 71.41 |
| Total | 100 | g | | 100 |

To make it easy for the chemist to prepare the formulation, the following is the same formula calculated for 1 gallon of bulk product:

TABLE 2

| ORIGINAL | | | POTENTIAL | ml or g |
|---|---|---|---|---|
| | | | Actives | |
| Sodium | 0.06521555 | | Natrum muriaticum | 652.16 |
| Camphor | 0.1431487 | | Camphora 3X | 143.15 |
| Menthol | 0.1431487 | | Mentholum 3X | 143.15 |
| Eucalyptol | 0.1431487 | | Eucalyptol 3X | 143.15 |
| | | | Total Actives | 1,081.60 |
| | | | Inactives | |
| Natural Sweet | 0.0837242 | | Natural Sweet Birch | 0.0837242 |
| Benzoin Resin | 0.0418621 | | Benzoin Resin | 0.0418621 |
| Thymol | 0.06968185 | | Thymol | 0.0696818 |
| Natural | 0.0215253 | | Natural Pine Oil | 0.0215252 |
| Pine Oil | | | | |
| Natural | 0.01431487 | | Cinnamon Oil | 0.0143148 |
| Spearmint Oil | 0.12490122 | | Spearmint Oil | 0.1249012 |
| Alcohol | 141.9375 | ml | Ethanol | 0 |
| Sodium | 0.04224060 | | Sodium | 0.042241 |
| Balance Water | 3,641.17 | g | Water | 71.41 |
| Total | 3785 | ml | | 2,703.00 |

The total of the 4 active ingredients (1,081.6 ml) are prepared using 261.98 ml of ethanol and 819.12 of water.

TABLE 3
The following is the formula which would
be needed for this bulk quantity:

| ORIGINAL | | | POTENTIAL | ml or g | |
|---|---|---|---|---|---|
| | | | Actives | | |
| Sodium | 3.58685525 | | Natrum | 35,868.552 | |
| | | | muriaticum | | |
| Camphor | 7.8731785 | | Camphora 3X | 7,873.1785 | |
| Menthol | 7.8731785 | | Mentholum 3X | 7,873.1785 | |
| Eucalyptol | 7.8731785 | | Eucalyptol 3X | 7,873.1785 | |
| | | | Total Actives | 59,488.88 | |
| | | | Inactives | | |
| Natural Sweet | 4.604831 | ml | Natural | 4.604831 | |
| | | | Sweet Birch | | |
| Benzoin Resin | 2.3024155 | | Benzoin Resin | 2.3024155 | |
| Thymol | 3.83250175 | | Thymol | 3.8325017 | |
| Natural Pine Oil | 1.18389123 | | Natural | 1.1838912 | |
| | | | Pine Oil | | |
| Natural | 0.78731785 | | Cinnamon Oil | 0.7873178 | |
| Spearmint Oil | 6.86956683 | | Spearmint Oil | 6.8695668 | |
| Alcohol | 7,806.5625 | | Ethanol | 0 | |
| Sodium | 2.323233 | g | Sodium | 2.323233 | g |
| Balance Water | 200,264.33 | | Water | 148,665 | ml |
| Total | 208,175 | ml | | 208,175 | ml |

The total of the 4 active ingredients (59,488.88 ml) are prepared using 14,409.1 ml of ethanol and 45,051.78 ml of water.

Although various ranges of formulation have been described above, various changes and modifications may be made by those skilled in the art for performing substantially the same function, in substantially the same way, to achieve substantially the same result, without departing from the true scope of the invention as defined in the appended claims.

Drawing Descriptions: No drawing description

1. A decongestant preparation comprising a set of active components comprising Natrum muriaticum, Camphora, Mentholum and Eucalyptol.

2. A decongestant of claim 1 wherein the total active components is about 28% by weight or volume of said decongestant preparation.

3. A decongestant of claim 1 wherein the active components are about:
17.23% Natrum muriaticum
3.78% Camphora
3.78% Mentholum
3.78% Eucalyptol
by weight or volume of said decongestant preparation.

4. A decongestant preparation of claim 1 wherein said decongestant preparation contains as additional one or more components.

5. A decongestant preparation of claim 1 wherein said decongestant preparation contains as additional components which is at least one member selected from the group consisting of Natural Sweet Birch Oil, Benzoin Resin, Thymol, Natural Pine Oil, Cinnamon Oil, Spearmint Oil, Sodium Bicarbonate, and Water.

6. A decongestant preparation of claim 5 wherein the components are about:
0.002212% Natural Sweet Birch Oil
0.001106% Benzoin Resin
0.001841% Thymol
0.0005687% Natural Pine Oil
0.0003782% Cinnamon Oil
0.0032999% Spearmint Oil
0.001116% Sodium Bicarbonate
71.41% Water
by weight or volume of said decongestant preparation.

7. A composition comprising:
about 17.23% Natrum muriaticum
about 3.78% Camphora
about 3.78% Mentholum
about 3.78% Eucalyptol
by weight or volume of said decongestant preparation.

8. A composition comprising:
about 17.23% Natrum muriaticum
about 3.78% Camphora
about 3.78% Mentholum
about 3.78% Eucalyptol
about 0.002212% Natural Sweet Birch Oil
about 0.001106% Benzoin Resin
about 0.001841% Thymol
about 0.0005687% Natural Pine Oil
about 0.0003782% Cinnamon Oil
about 0.0032999% Spearmint Oil
about 0.001116% Sodium Bicarbonate
about 71.41% Water
by weight or volume of said decongestant preparation.

9. A composition comprising:
17.23% Natrum muriaticum
3.78% Camphora
3.78% Mentholum
3.78% Eucalyptol
0.002212% Natural Sweet Birch Oil
0.001106% Benzoin Resin
0.001841% Thymol
0.0005687% Natural Pine Oil
0.0003782% Cinnamon Oil
0.0032999% Spearmint Oil
0.001116% Sodium Bicarbonate
71.41% Water
by weight or volume of said decongestant preparation.

10. A method of treating the occurrence of sinus pain and/or nasal congestion, comprising the step of administering to the subject a pharmaceutically effective amount of a composition comprising:
Natrum muriaticum
Camphora,
Mentholum
Eucalyptol
Natural Sweet Birch Oil
Benzoin Resin
Thymol
Natural Pine Oil
Cinnamon Oil
Spearmint Oil
Sodium Bicarbonate
Water

11. The method according to claim 10, wherein said subject is a mammal.

12. The method according to claim 11, wherein said subject is a human.

13. A method of inhibiting the occurrence of sinus pain and/or nasal congestion, comprising the step of administering to the subject a pharmaceutically effective amount of a composition comprising:
Natrum muriaticum
Camphora,
Mentholum
Eucalyptol
Natural Sweet Birch Oil
Benzoin Resin
Thymol
Natural Pine Oil
Cinnamon Oil
Spearmint Oil
Sodium Bicarbonate
Water

14. The method according to claim 13, wherein said subject is a mammal.

15. The method according to claim 14, wherein said subject is a human.

16. A method of reducing or alleviating respiratory problems comprising the step of administering to the subject a pharmaceutically effective amount of a composition comprising:
Natrum muriaticum
Camphora,
Mentholum
Eucalyptol
Natural Sweet Birch Oil
Benzoin Resin
Thymol
Natural Pine Oil
Cinnamon Oil
Spearmint Oil
Sodium Bicarbonate
Water

17. The method according to claim 16, wherein said subject is a mammal.

18. The method according to claim 17 wherein said subject is a human.

19. The method according to claim 16, wherein the respiratory problem is a sinus pain

20. The method according to claim 16, wherein the respiratory problem is a nasal congestion
